(19) United States
(12) Patent Application Publication
(10) Pub. No.: US 2020/0043161 A1
(43) Pub. Date: Feb. 6, 2020

(54) SYSTEM AND A METHOD FOR DETECTING A MATERIAL IN REGION OF INTEREST

(71) Applicant: The University of Western Australia, Crawley, Western Australia (AU)

(72) Inventor: Abraham J. Cense, Nedlands, Western Australia (AU)

(21) Appl. No.: 16/485,193

(22) PCT Filed: Feb. 16, 2018
(86) PCT No.: PCT/AU2018/050129

§ 371 Date: Aug. 12, 2019

Related U.S. Application Data
(60) Provisional application No. 62/460,384, filed on Feb. 17, 2017.

(51) Int. Cl. G06T 7/00; G06T 7/50; A61B 3/10; G01B 9/02

(52) U.S. Cl. CPC G06T 7/0012; G06T 7/50; A61B 3/102; G01B 2290/70; G01B 9/02091; G06T 2207/30041; G01B 9/02011

(57) ABSTRACT

An exemplary system, method and computer-accessible medium for detecting deposits in subjects can be provided, which can include, for example receiving information related to reference information obtained from measurements in further subjects, obtaining a threshold regarding a suspect data points in the information, and detecting the deposits in the subjects based on the threshold of the suspect data points. Retardances induced by the deposits can be located below the deposits using references that can be positioned above the deposits. Inner and outer segments of a photoreceptor layer can be used as a reference to collect the retardances located below a retinal pigment epithelium—Bruch's membrane complex.

### FIELD OF THE INVENTION

The present disclosure relates generally to a system and a method for detecting a material in region of interest and relates specifically, though not exclusively, to a system and method for detection of deposits within the region of interest and using polarization-sensitive optical coherence tomography (“PS OCT”).

### BACKGROUND

Currently, there is no method to pinpoint deposits in the retinal pigment epithelium—Bruch's membrane complex (“BM-RPE” complex). Scanning laser polarimetry (“SLP”) can be used to quantify polarization changes in the retina, in vivo, but since SLP only provides en face two-dimensional (“2D”) image quality, changes cannot be linked to a specific depth.

The present invention provides technological advancement.

### SUMMARY OF THE INVENTION

In a first aspect of the present invention there is provided a method for detecting a material in a region of interest, comprising:
providing at least one depth image of the region of interest, the region of interest being below a surface area of the region of interest and having a property;
processing the at least one depth image using a pre-determined threshold value of the property; and
detecting the material in the region of interest by analyzing the processed at least one depth image.

The method typically also comprises determining a threshold of the property. Determining the threshold of the property may comprise comparing depth images or depth image areas having the property, but which are not associated with the material with depth images or depth image areas having the property, but which are not associated with the material. In this case the depth images may be reference depth images of known regions of interest.

The step of processing the at least one depth image using the pre-determined threshold may comprise comparing the at least one obtained depth image with (a library of) reference depth images to determine if the obtained depth image has a property below or above the pre-threshold.

The step of processing the at least one depth image using the pre-determined threshold may comprise filtering out or discriminating against depth image areas or depth images for which the property is above or below the threshold of the property.

The step of detecting the material in the region of interest may comprise analyzing the processed at least one depth image.

The step of providing at least one depth image may comprise using OCT imaging of the region of interest. The OCT imaging may be PS OCT.

The material may be a deposit.

The step of providing the at least one depth image may be performed in-vivo or ex-vivo. In one example the step of providing the at least one depth image is performed in-vivo and the region of interest is a region of interest of a subject, such as a patient. The region of interest may be a region within an eye of the subject, such as the retinal pigment epithelium of a subject's eye and the deposit may be a deposit within the in the retinal pigment epithelium (such as BM-RPE).

For example, the property may be a local intensity of an area or pixels within the at least one depth image. In one specific example the property is a retardance of electromagnetic waves as detectable with PS OCT (for example, by determining a change in polarization angle relative to a reference).

The OCT imaging may be PS OCT and may use a reference at an interface with a deposit at a location below or above the deposit. In one specific example the deposit is a deposit in the retinal pigment epithelium—Bruch's membrane complex of a subject's eye. An inner or outer segment of a photoreceptor layer may be used as a reference.

The method may be conducted to detect deposits located in a retinal pigment epithelium—Bruch's membrane complex of a subject's eye (such as in the BM-RPE) by detecting associated retardance. Further, the method may be conducted to detect retardance induced by a further deposit that is located below another deposit using at least one reference that is positioned above the at least one the other deposit.

In a second aspect of the present invention there is provided a system for detecting a material in region of interest, the system being configured to:
provide at least one depth image of the region of interest, the region of interest being below a surface area of the region of interest;
process the at least one depth image using a pre-determined threshold value of the property; and
detect the material in the region of interest by analyzing the processed at least one depth image.

The system typically is also configured to determine the threshold of the property of associated with the at least one depth image.

In a third aspect of the present invention there is provided a non-transitory computer-accessible medium having stored thereon computer-executable instructions for detecting a material in region of interest, the computer arrangement being configured to perform procedures comprising:
providing at least one depth image of the region of interest, the region of interest being below a surface area of the region of interest;
determining a threshold of a property of associated with the at least one depth image;
processing the at least one depth image using the determined threshold; and
detecting the material in the region of interest by analyzing the processed at least one depth image.

The computer-accessible medium may be configured to utilize inner and outer segments of a photoreceptor layer as a reference to collect the at least one retardance located below a retinal pigment epithelium (such as BM-RPE).

In a fourth aspect of the present invention there is provided a non-transitory computer-accessible medium having stored thereon computer-executable instructions for detecting at least one deposit in at least one subject, wherein, when a computer arrangement executes the instructions, the computer arrangement is configured to perform procedures comprising:
receiving information related to a reference information obtained from measurements in at least one further subject;
obtaining a threshold regarding at least one suspect data point in the information; and
detecting the at least one deposit in the at least one subject based on the threshold of at least one suspect data point.

The computer arrangement may be further configured to detect at least one retardance induced by a further deposit that is located below or above the at least one deposit using at least one reference that is positioned above or below the at least one deposit. The computer arrangement may also be configured to utilize inner and outer segments of a photoreceptor layer as a reference to collect the at least one retardance for a region below a retinal pigment epithelium (such as BM-RPE). Further, the computer arrangement may be configured to determine the at least one deposit using at least one light source having a central wavelength. The central wavelength may be about 840 nm. The computer arrangement may be further configured to detect the at least one deposit using a polarization having adaptive optics.

In a fifth aspect of the present invention there is provided a system for detecting at least one deposit in at least one subject, comprising:
using a specifically configured computer hardware arrangement configured to:
   receive information related to a lookup table obtained from measurements in at least one further subject;
   determine a threshold of at least one suspect data point in the information; and
   detect the at least one deposit in the at least one subject based on the thresholded at least one suspect data point.

In a sixth aspect of the present invention there is provided a method for detecting at least one deposit in at least one subject, comprising:
receiving information related to a lookup table obtained from measurements in at least one further subject;
determine a threshold of at least one suspect data point in the information; and
using a specifically configured computer hardware arrangement, detecting the at least one deposit in the at least one subject based on the thresholded at least one suspect data point.

### BRIEF DESCRIPTION OF THE DRAWINGS

Features and advantages of the present invention will become apparent from the following description of embodiments thereof, by way of example only, with reference to the accompanying drawings in which:

FIG. 1 is a diagram of an exemplary spectral-domain Polarization-sensitive optical coherence tomography system according to an embodiment of the present invention;

FIGS. 2-3 are flow diagrams of a method of detecting a deposition according to an embodiment of the present invention;

FIG. 4 is a histogram illustrating data obtained using a method in accordance with an embodiment of the present invention;

FIGS. 5 and 6 are images illustrating data obtained using a method in accordance with an embodiment of the present invention;

FIGS. 7A-7D are images produced using the system and method according to an embodiment of the present invention;

FIGS. 8A-8K are further exemplary images produced using the system and method according to an embodiment of the present invention; and

FIG. 9 is an exemplary block diagram of a system in accordance with embodiments of the present invention.

### DETAILED DESCRIPTION OF EMBODIMENTS

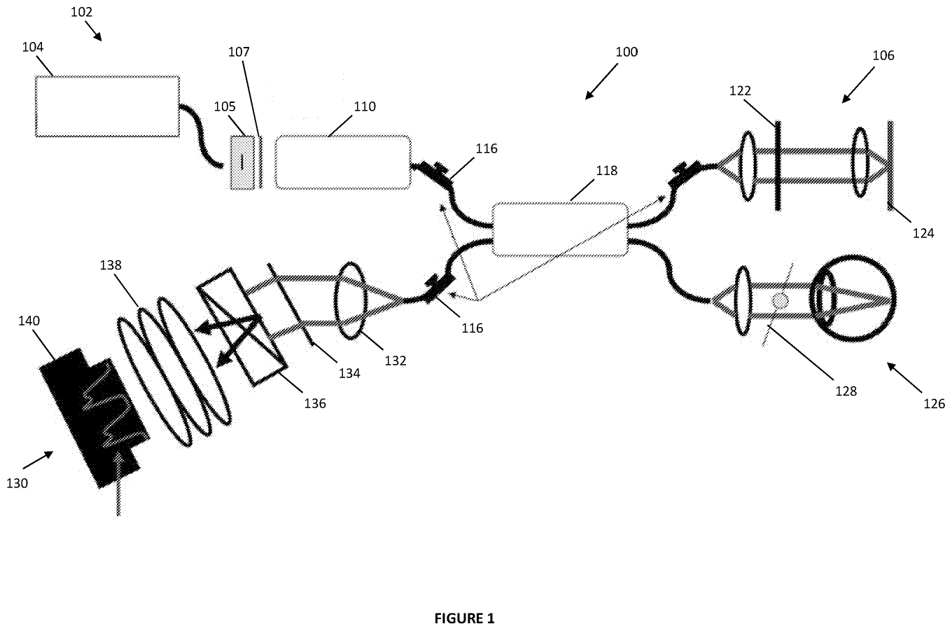

FIG. 1 shows a diagram of a spectral-domain PS-OCT system 100 according to an embodiment of the present disclosure. The system 100 has a source arm 102 with a broadband light source 104. Further, the system 100 has a reference arm 106. In this example the broadband light source 104 emits light within a wavelength range centered around (840 nm). In the source arm 102, light can be sent from the broadband light source 104 through an isolator 105 and two glass slides 107 for calibration. These glass slides can also be mounted in other arms of the interferometer, such as the reference arm 106. Information regarding the light can then proceed through a polarization modulator 110. The modulator 110 can apply two or more polarization states to the light, so that adjacent depth scans can be made with two orthogonal polarization states. Polarization controllers 116 are used to set the polarization state. The light is then split by a single-mode fiber coupler 118.

The reference arm 106 includes in this example a polarizer 122 and a mirror 124. The polarizer 122 is used in combination with the polarization controller 116 to ensure that the power returning to a detector can be the same for each polarization state, at each instant (e.g., even and odd depth scans, obtained with orthogonal input states).

The system 100 also has a sample arm 126 that includes in this example a raster scanning device 128 arranged to scan the retina of a subject.

The system 100 further has a detection arm 130. The detection arm 130 has in this example a polarization-sensitive high-speed spectrometer with a collimator 132, a transmission grating 134, a Wollaston prism 136, a focusing lens system 138 and line-scan camera 140. Detected spectra can be mapped to k-space, can be dispersion compensated, such that they can be Fourier transformed to depth without artifacts.

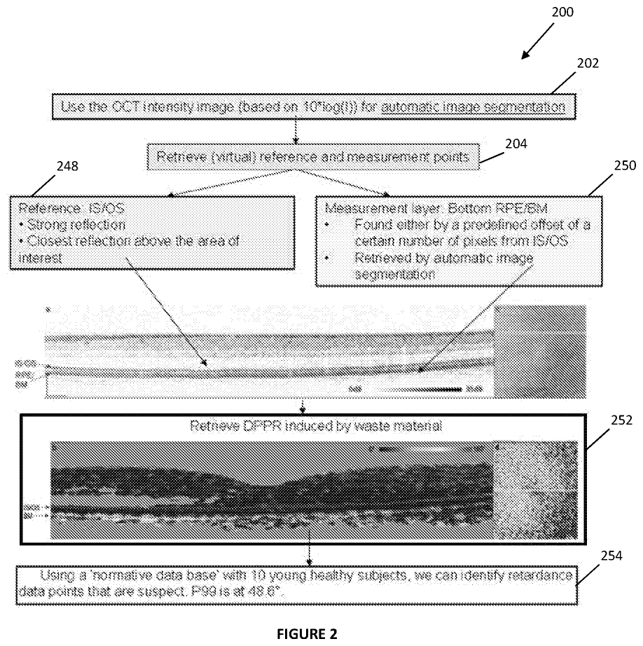

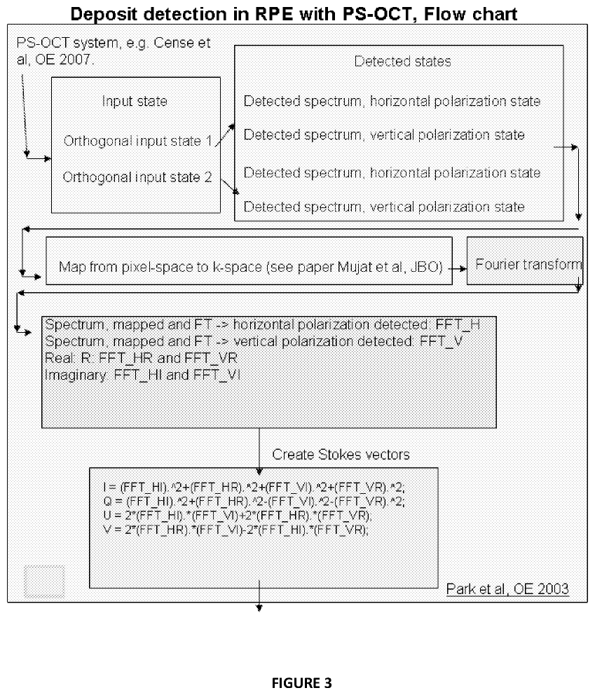

FIGS. 2 and 3 show flow diagrams of a method 200 of detecting a deposit in a subject's eye according to an embodiment of the present invention. The method 200 includes step 202, which uses OCT intensity images for automatic image segmentation of the inner and outer segments of the photoreceptor layer (“IS/OS”) and the bottom of the RPE-BM complex of the subject's eye. Step 204 retrieves (virtual) reference and measurement points and step 250 performs a measurement for the bottom layer of the RPE-BM complex. The bottom edge of this complex can be detected in healthy subjects and subjects with minor drusen by offsetting the IS/OS by 66 μm. Step 248 used the IS/OS as a reference, as it usually provides high reflectivity, and is the closest reflector to the deposits in the RPE-BM complex. A double pass phase retardation (“DPPR”) is calculated using Stokes-vector analysis with respect to the IS/OS as illustrated in flow chart shown in FIG. 3. Step 252 of the method 200 retrieves DPPR and collects the retardation induced by deposits at the bottom of the RPE/BM complex. The measured retardation at this depth includes contributions of the photoreceptor outer tips, RPE cells, BM and deposits in Bruch's membrane.

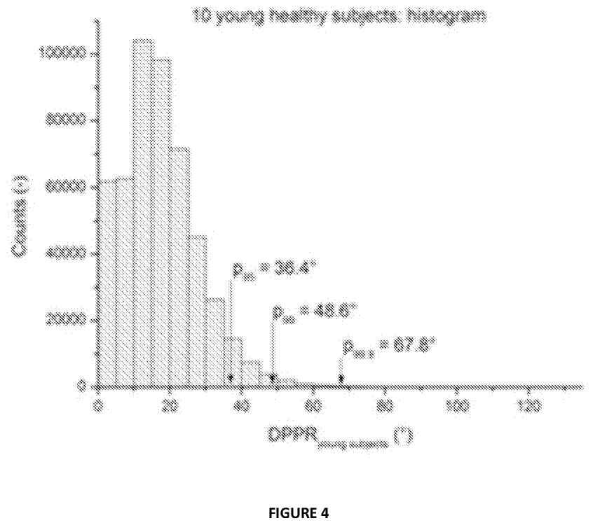

Step 254 of method 200 identifies retardance data points that are suspect using a “normal data base” with 10 young healthy suspects. FIG. 4 is a histogram illustrating the data obtained from the 10 healthy subjects. This data was used to find a threshold at which data points become suspect. For example, the 99^{th} percentile (e.g., about 48.6°) was used to identify these points. At p99, the 99^{th} percentile, a DPPR cut-off is only found to be in 1% of all data points.

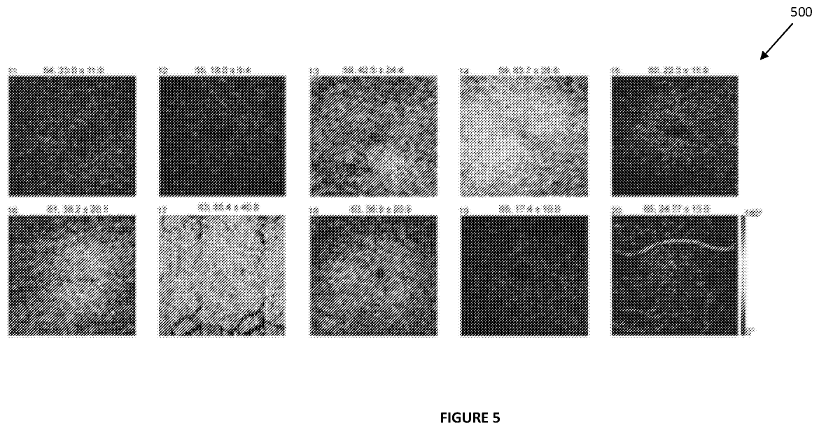

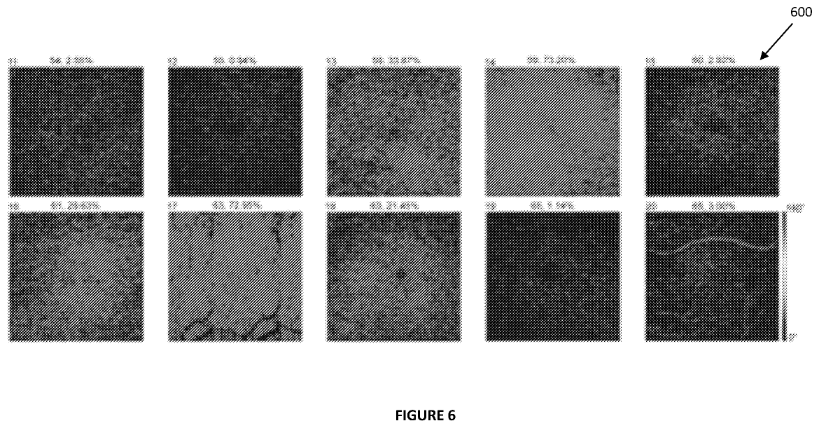

FIG. 5 illustrates the data 500 obtained from 10 older subjects; two of these subjects, for example, subjects 13 and 14 (see e.g., identifiers in the top left corner of each panel) were diagnosed as AMD patients; all other subjects did not have AMD. By applying a threshold at about 48.6°, suspect data points can be identified (see FIG. 6, identified points or pixels shown in lighter colour). While there were three other subjects with significantly elevated DPPR values these subjects were not diagnosed with AMD, showing that the exemplary method can identify data points that can be pre-clinical. These subjects likely have thin deposits in their RPE/BM complex that have not matured into drusen.

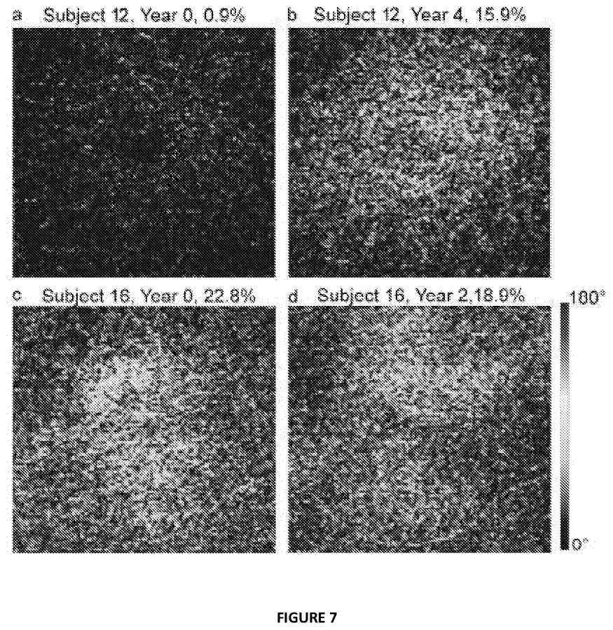

FIGS. 7A-7D show images produced using the system and method according to an embodiment of the present invention. The Figures show DPPR recorded below BM over about 15° by about 15° in a 59-year old subject in year 0 (e.g., FIG. 3A), and after one year (e.g., FIG. 3B). FIG. 7A illustrates data obtained in a 55-year old in year 0, and FIG. 7B shows the DPPR obtained after four years. The percentages above the images indicate how many data points were above the 99% cut-off of the 10 young subjects. The number of data points above the threshold increased with time, illustrating an accumulation of deposits.

FIGS. 7C and 7D show further exemplary images produced using the system and method according to an embodiment of the present invention. FIG. 7C illustrates data obtained in a 61-year old in year 0, with 22.8% of data points above the threshold, indicating accumulation of deposits. FIG. 7B shows the DPPR obtained after four years. Here, 19.8% of the data points were above the threshold.

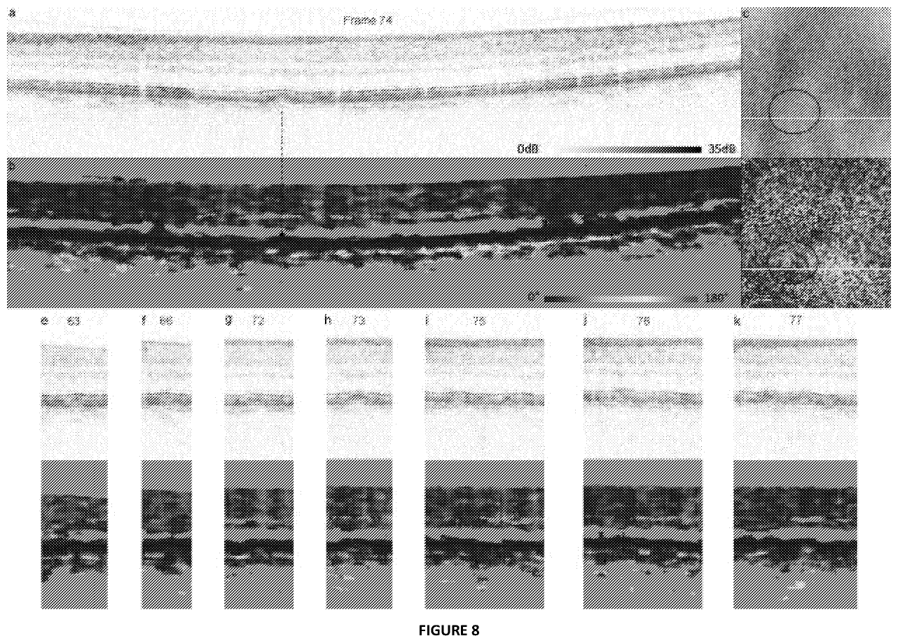

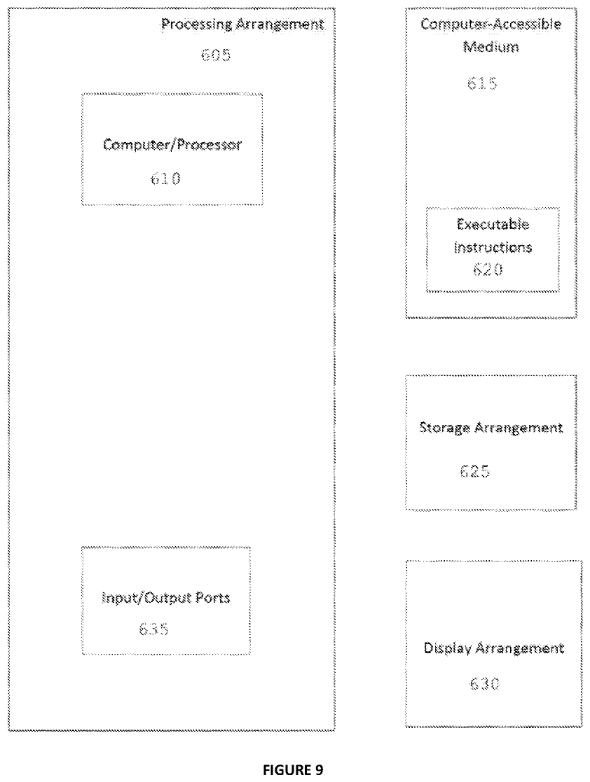

FIGS. 8A-8K show further images produced using the system and method according to an embodiment of the present invention. A druse can be found in the B-scan of subject 13 (e.g., FIG. 8A) in frame 74 (e.g., the volume set has 100 frames). The segmented IS/OS and RPE/BM are not shown, to better visualize the structure of the drusen. The edges of this approximately 150-μm wide druse (e.g., FIGS. 8A and 8B) generate moderate DPPR of approximately 90°, while the center generates little DPPR at the depth of the RPE/BM border (e.g., FIG. 8B). This can illustrate that the DPPR can be high when there can be little material between the RPE and BM, with decreasing DPPR with increased deposit thickness, due to hyalinization of the material. Similar observations were made for the other drusen in frames 63-77, within the area demarcated with the black ovals in FIGS. 8C and 8D, and also shown in FIGS. 8E-8K.

The use of PS-OCT for identifying deposits will now be summarized. Exemplary data was obtained with aPS-OCT system as described above with reference to FIGS. 1 to 3. The data was obtained on ten young healthy subjects and ten older subjects (e.g., 2-40). Two of these older subjects were diagnosed with AMD; all other subjects were graded as healthy without AMD.

The processing begins with k-space mapping and dispersion compensation. The real and imaginary parts of the acquired spectra can be converted into Stokes vectors. In this example each pixel in an image has four Stokes vectors I, Q, U and V. The retardance is calculated with respect to a reference.

The interface between the inner and outer segments of the photoreceptor layer (IS/OS) was used as a reference to avoid contamination of the retardance signal with contributions by the PS-OCT system itself, the cornea, retinal nerve fiber layer, and Henle fiber layer. A goal of the measurement was to quantify the retardance induced by the RPE-BM complex, which is located just below this reference.

The IS/OS reflect well, which provides a strong signal for reliable retardance calculation. The stronger reflections, the more reliable the DPPR calculation becomes. An objective is to detect polarization changes induced by deposits, which can occur in the RPE-BM complex, and it is beneficial to find a reference that can be close to these deposits, without being too close such that some spatial averaging can be applied. A reference that can be, for instance, at the top of the retina would not provide the same information, as birefringence contributions from the RNFL and the Henle fiber layer can contaminate the measurement that can be aimed at deposits in the RPE-BM complex. By using a reference at the top of the RNFL, the fast axis orientation of the RNFL can be used for retardance calculations, which can be different from the fast axis orientation of the tissue between the IS/OS and the RPE-BM complex, which can cause serious artifacts.

The IS/OS is beneficial for this purpose. The top of the RPE can also be used, but, as it is more closely spaced to the bottom of the RPE-BM complex, spatial averaging of Stokes vectors (which can be used to filter out speckle noise scrambling) can affect the retardance signal, as Stokes vectors at the top of the RPE can mix with Stokes vectors originating from the bottom of the RPE.

The IS/OS is in this example automatically segmented with image processing software.

Using the exemplary IS/OS as a reference, the retardance induced with respect to the IS/OS can be calculated. This is performed such that a color-coded image is generated, ranging from about 0° to about 180°, that is at about 0° at the IS/OS (see step 252 in FIG. 2). In the direction towards the RNFL, and in the direction towards the choroid, an increase in DPPR is observed. The bottom of the RPE-BM complex can be of interest, as it can provide the retardance induced by deposits in the RPE-BM complex, the RPE and the outer tips of the photoreceptor layer.

The bottom of the RPE-BM complex is found by an offset of about 66 μm in the downward direction from the segmented IS/OS. Automatic segmentation also provides the location of the bottom of the RPE-BM complex. The DPPR at this location can be collected and displayed in en face images.

Data was collected on ten young healthy subjects, and made a histogram of the DPPR, which shows the distribution of the number of pixels as a function of DPPR magnitude. This Gaussian-shaped histogram shows the relationship between DPPR and percentiles: the 99^{th} percentile can be at a DPPR of 48.6°; the 99.9^{th} percentile can be at 67.8°. Depending on how strictly the data of patients can be thresholded, a certain cut-off to threshold data can be applied.

Data was also collected on 10 older subjects (aged 54-65). By applying the 99^{th} percentile threshold to these data sets, data points that can be suspect can be identified. The suspect data points is set to magenta (in this example).

These maps can be used to monitor the development of deposits. An increase of DPPR with time shows that the RPE-BM complex can be plagued by deposits, which can lead to AMD. However, at this stage, there may not be an indication that the patient suffers from the disease, which can first manifest itself as drusen.

The exemplary elevated retardance values (e.g. above the 99^{th} percentile threshold of 48.6°) are in this example linked to deposits. For example, data sets obtained from two subjects, taken four and two years apart is shown in FIGS. 7A-7D. When comparing the data collected in year 0 with data collected in year 2 and year 4, there can be an increase in DPPR, which can be mainly concentrated in areas that already had the highest DPPR in year 0. Areas with deposits can be likely to block the flow of nutrients and waste material, thereby collecting more deposits over time.

Additionally, FIGS. 8A-8K illustrate the DPPR signal below the drusen. The drusen are comprised of “waste material”, and can look like mounds. The exemplary DPPR signal below the center of a druse is low, well below the 99^{th} percentile cut-off. At the edges of the druse, the DPPR signal is significantly higher. These images illustrate that when the collection of debris is still in an early stage, and the layer of debris is thin, such as at the edge of a druse, there is an elevated DPPR signal. In later stages, for example, when the subject already has AMD (the subject shown in FIGS. 8A-8K was confirmed with AMD), the DPPR signal drops to low values as the deposits is much thicker.

A distribution of the retardation that was recorded in the RPE-BM complex of ten young healthy subjects with spectral-domain PS-OCT at 840 nm is used to threshold data points that can be suspect, to identify areas with deposits in patients with age related macular degeneration or patients who are about to have age related macular degeneration.

As mentioned above, a reference for polarization-sensitive analysis can be located at the automatically segmented interface between the inner and outer segments of the photoreceptor layer (IS/OS); the retardance induced by deposits is recorded at or below the RPE-BM complex, which can either be found by an offset from the IS/OS, or by automatic image segmentation. The reference can alternatively also be located at the top of the RPE (towards the RNFL). A reference higher up in the retina, for example, at the top of the RNFL can also be used as a reference. While it can provide less accurate measurements, it can still provide some signal in the RPE-BM complex. This can benefit from a re-calibration of the data set illustrated in FIG. 4, to determine the cut-off for an accurate threshold to detect deposits.

A spectral-domain system at any other central wavelength than 840 nm can be used (e.g. visible range, or near infra-red range near 1050 nm). Further, a swept-source PS-OCT system at any wavelength can be used. The exemplary system can be utilized with adaptive optics.

FIG. 6 shows a block diagram of an embodiment of a system according to the present invention. Procedures can be performed by a processing arrangement and/or a computing arrangement 605. Such processing/computing arrangement 605 can be, for example entirely or a part of, or include, but not limited to, a computer/processor 610 that can include, for example one or more microprocessors, and use instructions stored on a computer-accessible medium (e.g., RAM, ROM, hard drive, or other storage device).

As shown in FIG. 6, for example a computer-accessible medium 615 (e.g., as described herein above, a storage device such as a hard disk, floppy disk, memory stick, CD-ROM, RAM, ROM, etc., or a collection thereof) can be provided (e.g., in communication with the processing arrangement 605). The computer-accessible medium 615 can contain executable instructions 620 thereon. In addition or alternatively, a storage arrangement 625 can be provided separately from the computer-accessible medium 615, which can provide the instructions to the processing arrangement 605 so as to configure the processing arrangement to execute exemplary procedures, processes and methods, as described herein above.

Further, the exemplary processing arrangement 605 can be provided with or include an input/output arrangement 635, which can include, for example a wired network, a wireless network, the internet, an intranet, a data collection probe, a sensor, etc. As shown in FIG. 6, the exemplary processing arrangement 605 is in communication with an exemplary display arrangement 630, which, can be a touch-screen configured for inputting information to the processing arrangement in addition to outputting information from the processing arrangement, for example. Further, the exemplary display 630 and/or a storage arrangement 625 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

The following references are hereby incorporated by reference in their entireties:

[1] Cense B, Wang Q, Lee S, Zhao L, Elsner A E, Hitzenberger C K, Miller D T. Henle fiber layer phase retardation measured with polarization-sensitive optical coherence tomography. Biomedical optics express. 2013 Nov. 1; 4(11):2296-306.

[2] Mujat M, Park B H, Cense B, Chen T C, de Boer J F. Autocalibration of spectral-domain optical coherence tomography spectrometers for in vivo quantitative retinal nerve fiber layer birefringence determination. Journal of biomedical optics. 2007 July; 12 (4):041205.

[3] Chiu S J, Li X T, Nicholas P, Toth C A, Izatt J A, Farsiu S. Automatic segmentation of seven retinal layers in SDOCT images congruent with expert manual segmentation. Optics express. 2010 Aug. 30; 18(18):19413-28.

[4] Park B H, Pierce M C, Cense B, de Boer J F. Real-time multi-functional optical coherence tomography. Optics express. 2003 Apr. 7; 11(7):782-93.

[5] Cense B, Chen T C, Park B H, Pierce M C, de Boer J F. In vivo depth-resolved birefringence measurements of the human retinal nerve fiber layer by polarization-sensitive optical coherence tomography. Optics letters. 2002 Sep. 15; 27(18):1610-2.

[6] Cense B, Chen T C, Park B H, Pierce M C, De Boer J F. Thickness and birefringence of healthy retinal nerve fiber layer tissue measured with polarization-sensitive optical coherence tomography. Investigative ophthalmology & visual science. 2004 Aug. 1; 45(8):2606-12.

[7] Park B H, Pierce M C, Cense B, de Boer J F. Optic axis determination accuracy for fiber-based polarization-sensitive optical coherence tomography. Optics letters. 2005 Oct. 1; 30(19):2587-9.

1. A method for detecting a material in region of interest, comprising:
providing at least one depth image of the region of interest, the region of interest being below a surface area of the region of interest and having a property;
processing the at least one depth image using a pre-determined threshold value of the property; and
detecting the material in the region of interest by analyzing the processed at least one depth image.

2. The method of claim 1 comprising determining a threshold of the property.

3. The method of claim 2 wherein determining the threshold of the property comprises comparing depth images or depth image areas having the property and which are associated with, or show, the material with depth images or depth image areas having the property, but which are not associated with the material.

4. The method of claim 3 wherein the depth images are reference depth images of a known region of interest.

5. The method of claim 1 wherein the step of processing the at least one depth image using the pre-determined threshold comprises comparing the at least one obtained depth image with at least one reference depth images to determine if the obtained depth image has a property below or above the pre-threshold of the property.

6. The method of claim 1 wherein the step of processing the at least one depth image using the determined threshold comprises filtering out or discriminating against depth image areas or depth images for which the property is above or below the pre-determined threshold of the property.

7. The method of claim 1 wherein the step of providing at least one depth image comprises using PS OCT imaging of the region of interest.

8. The method of claim 1 wherein the material is a deposit.

9. The method of claim 1 wherein the step of providing the at least one depth image is performed in-vivo.

10. The method of claim 1 wherein the step of providing the at least one depth image is performed in-vivo and the region of interest is a region of interest of a subject.

11. The method of claim 10 wherein the region of interest is a region within an eye of the subject.

12. The method of claim 11 wherein the region of interest is a region in the retinal pigment epithelium of a subject's eye and the deposit is a deposit within the in the retinal pigment epithelium.

13. The method of claim 1 wherein the property is a local intensity of an area or pixel within the at least one depth image.

14. The method of claim 1 wherein the property is a retardance of electromagnetic waves as directly or indirectly detectable with PS OCT.

15. The method of claim 14 wherein the PS OCT uses a reference at an interface with a deposit at a location below or above the deposit.

16. The method of claim 15 wherein the deposit is a deposit in the retinal pigment epithelium of a subject's eye.

17. The method of claim 16 wherein an inner or outer segment of a photoreceptor layer is used as a reference.

18. The method of claim 1 wherein the method is conducted to detect deposits located in a retinal pigment epithelium of a subject's eye by detecting associated retardance(such as BM-RPE).

19. A system for detecting a material in region of interest, the system being configured to:
provide at least one depth image of the region of interest, the region of interest being below a surface area of the region of interest;
process the at least one depth image using a pre-determined threshold value of the property; and
detect the material in the region of interest by analyzing the processed at least one depth image.

20. The system of claim 19 wherein the system is also configured to determine the threshold of the property of associated with the at least one depth image.
